(19) United States
(12) Patent Application Publication
(10) Pub. No.: US 2011/0288577 A1
(43) Pub. Date: Nov. 24, 2011

(54) APPARATUS AND METHOD FOR JOINING ADJACENT TISSUE

(75) Inventors: Richard R. Newhauser, Redwood City, CA (US); Travis R. Yribarren, Campbell, CA (US)

(73) Assignee: ABBOTT CARDIOVASCULAR SYSTEMS, INC., Santa Clara, CA (US)

(21) Appl. No.: 12/782,392

(22) Filed: May 18, 2010

(51) Int. Cl. A61M 29/00

(52) U.S. Cl. 606/195

(57) ABSTRACT

Apparatuses and methods are disclosed for reducing the size of openings within a heart valve or other opening in the wall of a body lumen by implanting an elastically expanded member around the opening and then permitting the expandable member to contract. The expandable member may encircle a portion of an inflatable member, the distal end of which is inserted through the opening along with the expandable member. The inflatable member is inflated to expand the expandable member and then drawn proximally form the opening to drive projections formed on the expandable member into the valve or wall. The inflatable member is then withdrawn, allowing the expandable member to elastically contract. The inflatable member may have first and second stages, where the second stage is distal of the first stage and has a larger inflated diameter.

### BACKGROUND OF THE INVENTION

1. The Field of the Invention

This application relates generally to systems and method for percutaneously associating adjacent tissue, and more particularly for repairing mitral valve abnormalities.

2. The Relevant Technology

Referring to FIGS. 1A and 1B, the mitral valve 10 of the heart 12 prevents the flow of blood from the left ventricle 14 into the right atrium 16 during ventricular systole. The mitral valve 10 includes two leaflets 18a, 18b that spread to permit blood flow into the left ventricle 14 during ventricular diastole and are forced together by pressure within the left ventricle 14 during ventricular systole. Degeneration of the mitral valve 10 or surrounding tissue may result in a gap 20 between the leaflets 18 during ventricular systole, resulting in regurgitation of blood into the left atrium. When severe, mitral regurgitation can lead to heart failure and abnormal heart rhythms.

Current methods of repairing the mitral valve 10 include the joining of the valve leaflets 18a, 18b at one or more locations to decrease the flow cross section and prevent valve regurgitation. This method is generally referred to as the Alfieri method. Currently known devices that permit the Alfieri method to be practiced percutaneously include devices for gripping the valve leaflets 18a, 18b and suturing them together. However, these percutaneous methods face considerable drawbacks due to the difficulty in grasping the valve leaflets due to their rapid movement. The methods also require adequate visualization of the valve which is difficult to achieve. Available visualization techniques using fluoroscopy can be difficult to use, since a sufficient bolus of contrast is difficult to administer. Echocardiography is likewise unable to provide adequate visualization of the mitral valve.

### BRIEF SUMMARY OF THE INVENTION

These and other limitations are overcome by embodiments of the disclosure, which relates to apparatuses and methods for repairing a mitral valve, patent foramen ovale, or a puncture site in a blood vessel or wall of another body lumen are disclosed. In particular, apparatuses and methods are disclosed for inserting a portion of an inflatable member encircled by an elastically expandable member through the mitral valve, or other opening in a wall of a body lumen. The elastically expandable member bears projections adapted to penetrate the wall or valve. The inflatable member may be inflated to expand the expandable member. The inflatable member may then be drawn through the valve or opening such that the projections are driven into the valve or opening. The inflatable member may then be withdrawn and the expandable member allowed to contract in order to reduce the size of the opening in the valve or wall. The inflatable member may be deflated either before or after withdrawal.

In one aspect of the invention, the elastically expandable member includes a thin circuitous member defining a base line lying in a plane and circumscribing the inflatable member. The thin circuitous member may be bent to define a first plurality of peaks extending a first distance measured from the base line parallel to the inflatable member and a second plurality of peaks extending a second distance measured from the base line parallel to the inflatable member, the second distance being substantially greater than the first distance.

In another aspect of the invention, a catch is secured adjacent the inflatable member and has a diameter greater than that of an adjacent portion of the elastically expandable member when the inflatable member is not inflated.

In another aspect of the invention, the inflatable member includes a first portion and a second portion, the elastically expandable member encircling the first portion and the second portion having an inflated diameter substantially greater than the first portion.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims.

### BRIEF DESCRIPTION OF THE DRAWINGS

To further clarify some of the advantages and features of the present invention, a more particular description of the invention will be rendered by reference to specific embodiments thereof which are illustrated in the appended drawings. It is appreciated that these drawings depict only illustrated embodiments of the invention and are therefore not to be considered limiting of its scope. The invention will be described and explained with additional specificity and detail through the use of the accompanying drawings in which:

FIG. 1A is a partial cutaway view of a heart;

FIG. 1B is an illustration of a mitral valve;

FIG. 2 is a side view of a device for implanting an elastically expandable member in accordance with an embodiment of the present invention;

FIG. 3 is an isometric view of the device for implanting an elastically expandable member positioned within a mitral valve in accordance with an embodiment of the present invention;

FIG. 4 is a schematic diagram of an inflation system for use with the device for implanting an elastically expandable member in accordance with an embodiment of the present invention;

FIGS. 5A and 5B are isometric views of an elastically expandable member in accordance with an embodiment of the present invention;

FIG. 6 is a partial side view of the device for implanting an elastically expandable member in accordance with an embodiment of the present invention;

FIG. 7 is a front end view of the device for implanting an elastically expandable member in accordance with an embodiment of the present invention;

FIGS. 8A through 8F illustrate a method for implanting an elastically expandable member in accordance with an embodiment of the present invention;

FIG. 9 is a side view of a device for implanting an elastically expandable member having multiple inflatable portions in accordance with an embodiment of the present invention;

FIG. 10 is a schematic diagram of an inflation system for use with the device of FIG. 9 in accordance with an embodiment of the present invention;

FIGS. 11A through 11C illustrate another method for implanting an elastically expandable member in accordance with an embodiment of the present invention;

FIGS. 12A through 16B illustrate a method for implanting an elastically expandable member in an opening within a wall of a body lumen in accordance with an embodiment of the present invention;

FIG. 17 illustrates an alternative embodiment of an elastically expandable member in accordance with an embodiment of the present invention; and

FIGS. 18A through 18C illustrate a method for implanting the elastically expandable member of FIG. 17.

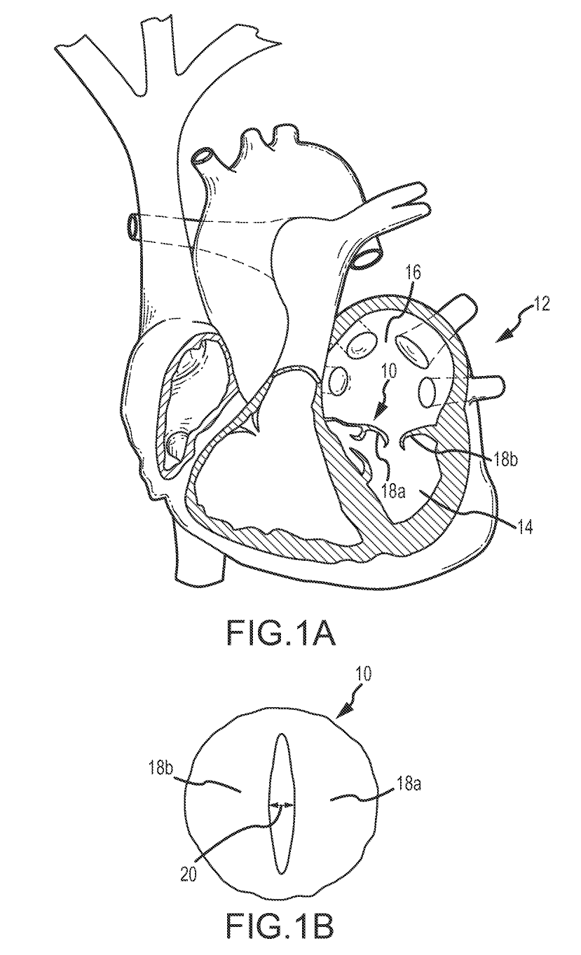

### DETAILED DESCRIPTION OF THE PREFERRED EMBODIMENTS

Embodiments of the invention relate to associating adjacent tissue including joining adjacent tissue. Tissue can be brought together by an expandable medical device that can be expanded during deployment. The device includes attachment means to grasp or connect to the adjacent tissue. The medical device then collapses to reduce in profile and bring adjacent tissue together. By way of example only, embodiments of the invention can be used to repair mitral valves, perform vessel closure, perform PFO (Patent Foramen Ovale) closure, and repair other septal defects.

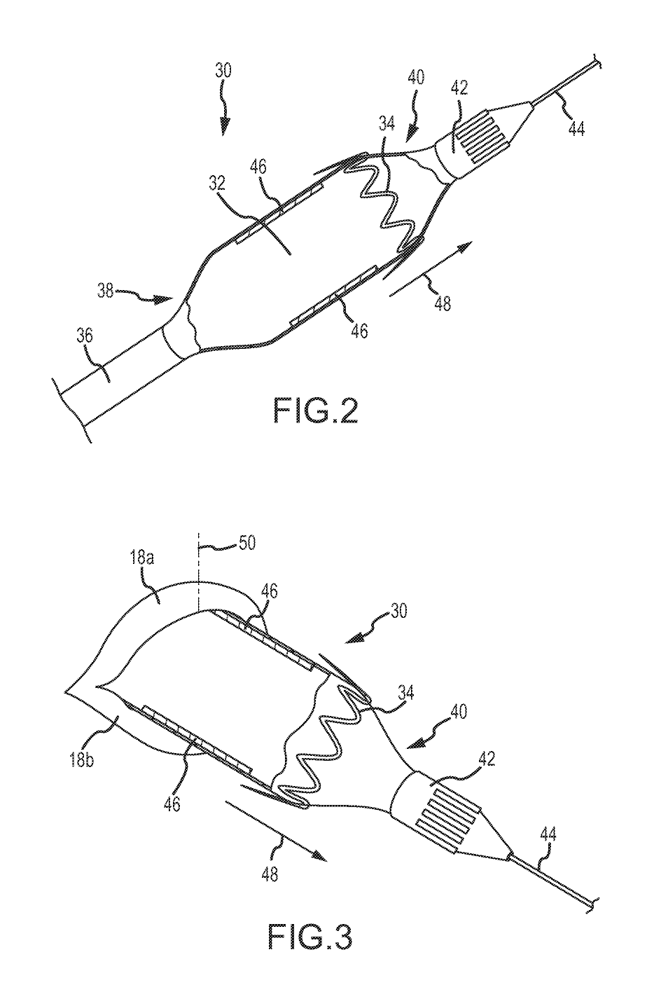

Referring to FIG. 2, an apparatus 30 for repairing a mitral valve may include an inflatable member 32, an elastically expandable member 34 encircling or disposed around the inflatable member 32, and a catheter 36. The inflatable member 32 includes a distal end 40 and a proximal end 38 secured to the catheter. An inflation medium may be delivered through the catheter 36 to the inflatable member through the proximal end 38. The inflatable member 32 may have an uninflated diameter less than that of the lumen of the catheter 36.

A safety catch 42 may be secured near the distal end of the inflatable member 32 and prevent the expandable member 34 from sliding off of the inflatable member before placement as described hereinbelow. In some embodiments, the safety catch may cover a distal end 40 of the inflatable member 34. The safety catch 42 may include a structure having an outer diameter greater than an undeformed outer diameter of the immediately adjacent portion of the inflatable member 34 to which it secures, such as near the distal end 40 thereof. For example, at least a length of the inflatable member 32 immediately adjacent the safety catch 42 may have a relaxed outer diameter less than the outer diameter of the safety catch. The length may be at least half as wide as the expandable member 34 along the longitudinal axis of the inflatable member 32. The safety catch 42 preferably includes a structure having an outer diameter greater than an undeformed inner diameter of the expandable member 34. Alternatively, the safety catch 42 has an outer diameter greater than an uninflated diameter of the inflatable member 32. In the illustrated embodiment, the safety catch 42 is a cylindrical structure. However, other shapes having different cross section may be used having at least a portion therefore with a diameter greater than either the undeformed inner diameter of the expandable member 34 or the uninflated diameter of the inflatable member 32.

The inflatable member 32 may define a lumen for receiving a guide wire 44. The guide wire 44 may be guided to an operation site as known in the art. The catheter 36 and inflatable member 32 may then be guided along the guide wire 44 to the operation site. In some embodiments, the inflatable member 32 is positioned within the catheter 36 during insertion proximate the distal end 40 thereof during insertion of the catheter. In other embodiments, the catheter 36 is first inserted to the operation site after which the inflatable member 32 and expandable member 34 are fed through the catheter 36 to the operation site.

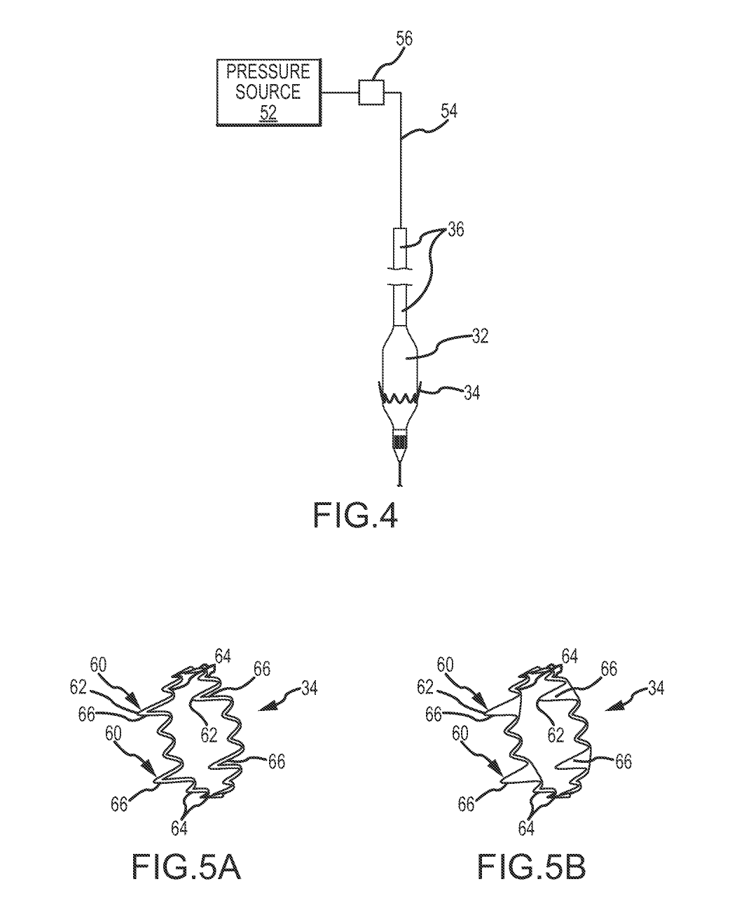

Referring to FIG. 3, while still referring to FIG. 2, the inflatable member 32 may bear markers 46 including a radiopaque material. Examples of biocompatible radiopaque materials include platinum, iridium, tungsten, tantalum, gold, and alloys thereof. In an alternative embodiment, the markers 46 are designed to be highly visible using echocardiography, such as by forming the markers 46 of a very dense material. The markers 46 may be elongate in shape having a direction of elongation extending parallel to a distal direction 48 in which the inflatable member 32 is moved during placement in the operation site as described hereinbelow.

During placement in a mitral valve 10 the radiopaque markers 46 are approximately aligned with a midline 50 of the mitral valve 10 using fluoroscopy or other visualization technique. For example, the inflatable member 32 may be inserted within the mitral valve 10 and then rotated into the position shown in FIG. 3. Alternatively, the markers 46 may be positioned on or adjacent the midline 50 prior to insertion such that rotation is not necessary.

As is readily apparent, the high visibility of the markers 46 using fluoroscopic or echocardiographic imaging enables ready positioning of the inflatable member 32 without the need for detailed imaging of the mitral valve 10. The opening between the leaflets 18a, 18b of the mitral valve 10 may be readily apparent using echocardiography or fluoroscopy equipment. The high visibility of the markers 46 therefore enables them to be readily aligned such that they both lie on a midline 50 perpendicular to the opening between the leaflets 18a, 18b.

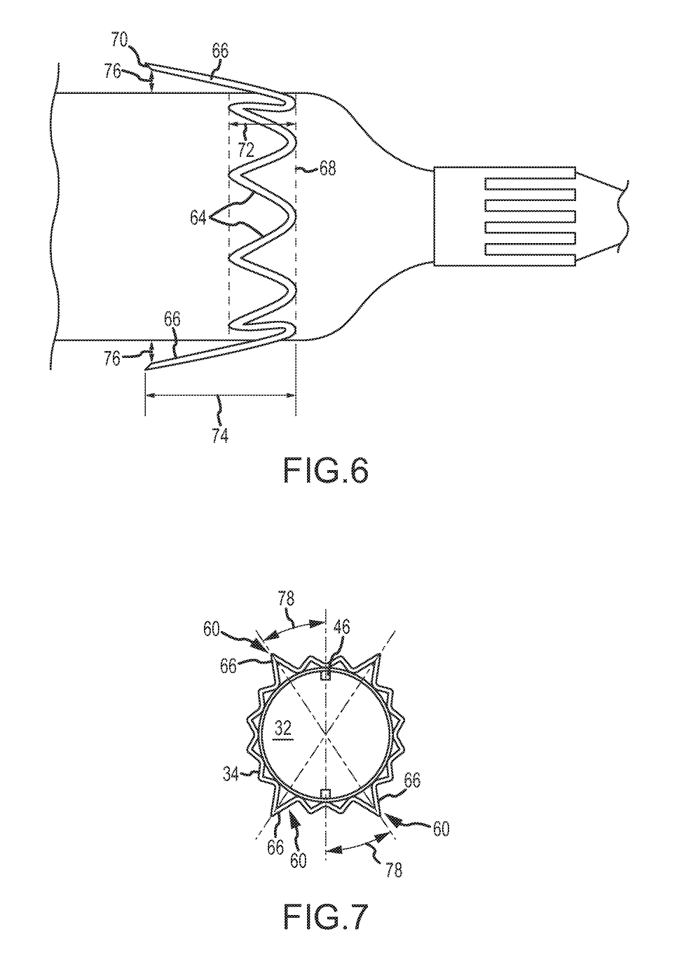

Referring to FIG. 4, inflatable member 32 may be inflated by means of a pressure source 52 external to the patient's body and coupled to the inflatable member 32 by means of a tube 54 extending from the pressure source 52, through the catheter 36, and to the inflatable member 32. In some embodiments, the lumen 36 of the catheter provides a seal with the proximal end 38 of the inflatable member, such that a separate tube 56 is not needed within the catheter 36. An inflation medium supplied from the source 52 may be controlled by a valve 56 that is selectively opened by the operator to inflate the inflatable member according to the method described hereinbelow. The valve 56 may also control selective release of the inflation medium from the inflation member 32. In accordance with this invention, the inflation medium provided by pressure source 52 may be a gas or a liquid. For example, it may be any biocompatible gas, such as carbon dioxide, that is typically used in medical procedures, or it may be a fluid, such as saline, which also is also widely used in medical procedures such as those contemplated in this disclosure.

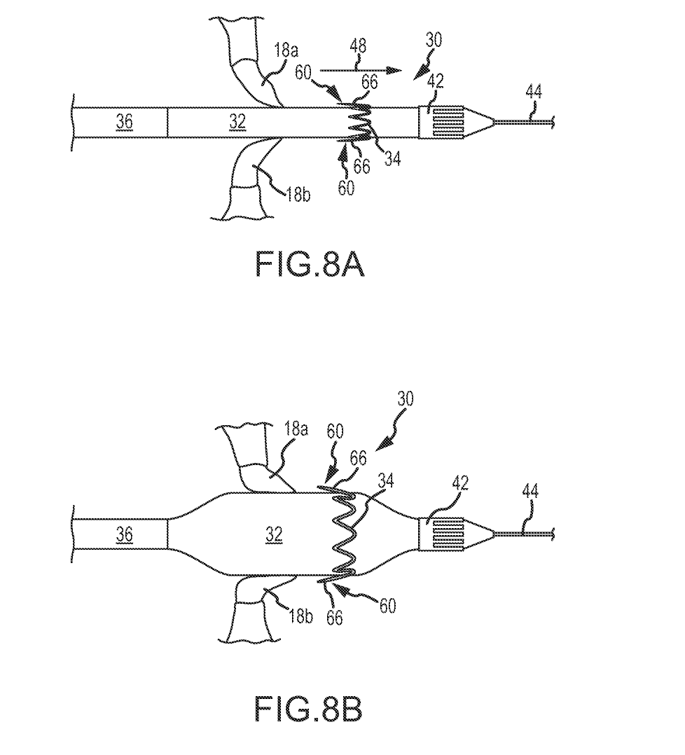

Referring to FIG. 5A, the expandable member 34 encircles the inflatable member 32 and elastically deforms as the inflatable member 32 is inflated. The expandable member 34 includes one or more projections 60 adapted to pierce the leaflets 18a, 18b of the mitral valve 10. Accordingly, the projections 60 may have a converging profile such that the ends 62 of the projections 60 or of selected projections 60 are sharp enough to readily penetrate the leaflets 18a, 18b.

In the illustrated embodiment, the expandable member 34 is formed from a thin elastic material bent or shaped to form first peaks 64 and second peaks 66. For example, the expandable member 34 may be formed from a resilient wire including a material such as nitinol.

Referring to FIG. 5B, in some embodiments the peaks 66 are not themselves expandable in order to reduce tissue spreading when the expandable member 34 is inserted into the leaflets 18a, 18b. As shown in FIG. 5B, in such embodiments, the peaks 66 may be secured to the expandable member due to monolithic formation therewith or by means of welding. Alternatively, peaks 66 may be formed by undulations in the expandable member that have been welded or otherwise fastened to prevent spreading of the peaks 66 when the expandable member 34 is expanded.

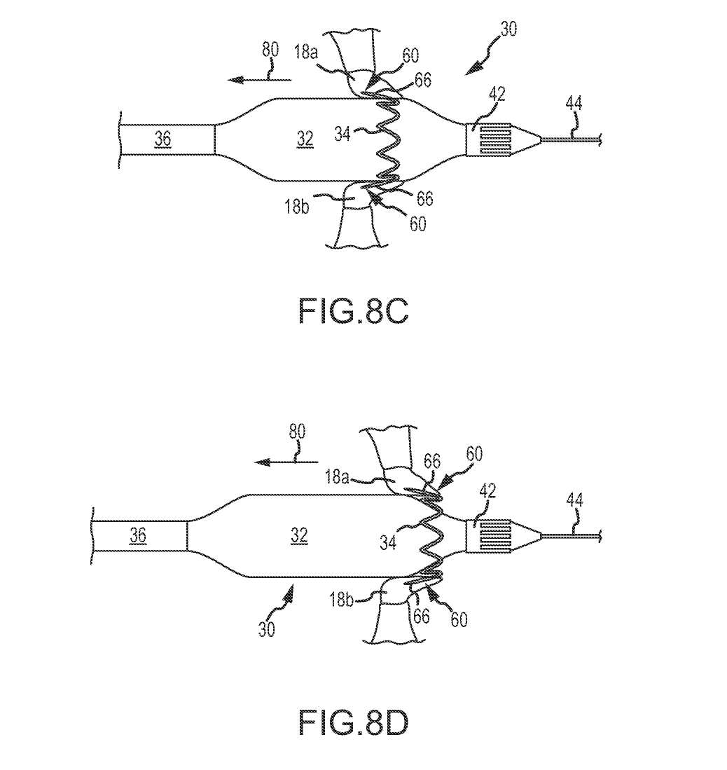

Referring to FIG. 6, the second peaks 66 may be longer than the first peaks 64 and serve as the projections 60 for piercing the leaflets 18a, 18b of the mitral valve or of the tissue to be closed, joined, or associated. The second peaks 66 may include one or more beveled edges 70 at their ends to provide a sharper point for penetrating the leaflets 18a, 18b or for penetrating other tissue, such as vessel walls by way of example only. The second peaks 66 may be separated from one another by means of one or more first peaks 64. The expandable member 34 may define a base line 68, which is a base circle 68 in the illustrated embodiment. However, the base line 68 may have any shape forming a closed loop. The base line 68 lies in a plane perpendicular to the longitudinal axis of the inflatable member 32, or in other words perpendicular to the distal direction 48.

The first peaks 64 may extend up to a first distance 72 from the base circle 68 and the second peaks 66 may extend at least a second distance 74 from the base circle 68. The second distance 74 may be substantially greater than the first distance 72. For example, the second distance 74 may be between 1.1 and 2 times the first distance 72, or between about 1.2 and 1.6 times the first distance 72. The second distance 74 may also be measured relative to the diameter of base circle 68. For example, second distance 74 may be between about 0.3 and 0.8 of the diameter of base circle 68, although any ratio may be contemplated within this invention.

The second peaks 66 may also project away from the inflatable member 32 with distance from the base circle 68. For example, the second peaks 66 may define an angle 76 with respect to the inflatable member that is between 5 and 25 degrees, or between about 5 and 15 degrees. The angled second peaks 66 enable the member to engage the tissue to be joined by providing a separation between the second peaks 66 and the inflatable member 32. The angle 76 may also change as the expandable member is expanded by the inflatable member.

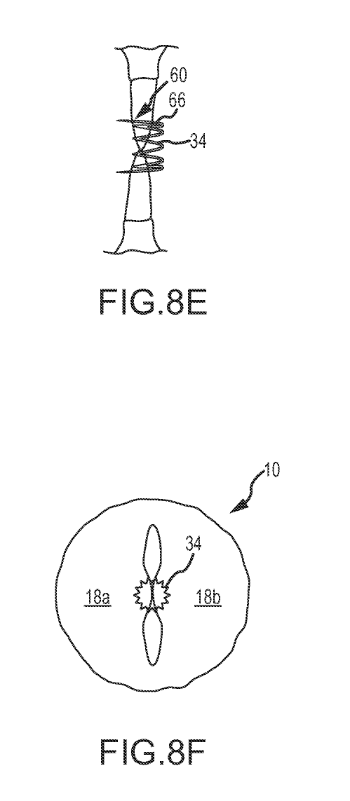

Referring to FIG. 7, in the illustrated embodiments, the second peaks 66 are positioned an angular distance 78 from the markers 46 such that they will be adjacent to the leaflets 18a, 18b when the markers 46 are aligned with the midline 50 of the mitral valve 10. For example, projections 60 may be within an angular distance 78 from the marker 46 equal to between about 5 and 30 degrees, or between about 5 and 15 degrees. In the illustrated embodiment, at least two of the projections 60 engage each leaflet 18a, 18b. However, in other embodiments, at least three or more projections 60 may engage each leaflet 18a, 18b. In some embodiments, some of the first peaks 64 may also engage the tissue in certain instances.

For example, the device 30 illustrated in FIGS. 2-7 depict an expandable medical device 30 in both an expanded an unexpanded state. In an unexpanded state, the device is positioned at an operational site, such as at a mitral valve. The device 30 can include a generally tubular body formed from a material such as a shape memory material as described below. The device 30 may comprise a ring that may be circular in shape. One will appreciate that the device 30 can include a pattern or configuration that permits the device 30 to be placed into a delivery configuration having a first diameter and radially expanded to at least a deployment configuration having a second, larger diameter as explained in greater detail below. In one embodiment, the first diameter corresponds to an unconstrained configuration of the device 30 and the device 30 may be configured to return to the unconstrained configuration after being subject to a deforming force, such as applied by the inflatable member to expand the device 30 during deployment.

The device 30 can have a serpentine or undulating configuration formed from a plurality of peaks and corresponding valleys. As previously described, some of the peaks are longer than other peaks. As illustrated in FIG. 5, for example, the projections 60 may correspond to peaks that are longer that other peaks. These peaks are also configured to engage tissue in order to bring adjacent tissue closer together. The serpentine or undulating configuration can be sinusoidal, triangular, square, and the like. In some instances, the serpentine or undulating configuration can include multiple shapes that are repeated in series and/or have a similar shape that varies in size.

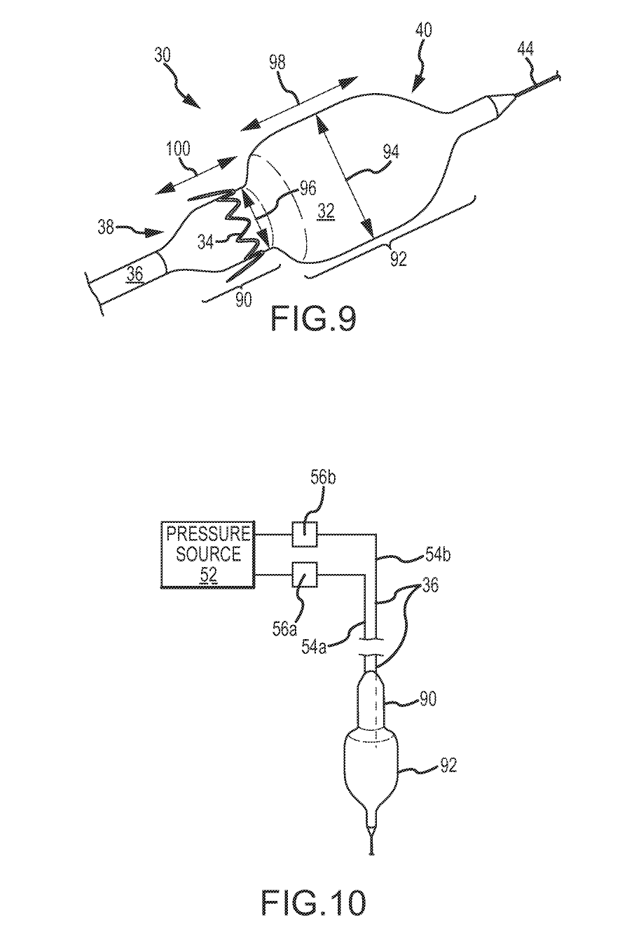

A device 30 such as is described with respect to FIGS. 2 through 7, may be used in accordance with a method illustrated in FIGS. 8A through 8F. Referring specifically to FIG. 8A, a portion of the inflatable member 32 encircled by the expandable member 34 is urged through the mitral valve 10 into the left ventricle 14 along the distal direction 48 such that the proximal end 38 and distal end 40 of the inflatable member 32 are positioned on opposite sides of the mitral valve 10. In some embodiments, the inflatable member 32 is extended from the catheter 36 prior to insertion through the mitral valve 10. In other embodiments, the catheter 36 is inserted through the mitral valve 10 and then withdrawn as the inflatable member 32 is forced out of the catheter 36, leaving the inflatable member in the position shown in FIG. 8A.

Referring to FIG. 8B, the inflatable member 32 may then be inflated in order to expand the expandable member 34. Expansion of the inflatable member 32 may also aid in positioning of the leaflets 18a, 18b of the mitral valve 10, which would otherwise be prone to rapid movement responsive to beating of the heart 12. In some embodiments inflation of the inflatable member 32 results in an inflated diameter that is between 1.5 and three times either the uninflated diameter of the inflatable member 32 or the inner diameter of the lumen of the catheter 36. In one embodiment, the inflated diameter is between about two and three times either the uninflated diameter of the inflatable member 32 or the inner diameter of the catheter 36.

Referring to FIG. 8C, the inflatable member 32 and expandable member 34 may then be urged in proximal direction 80 such that at least some of the projections 60 penetrate the leaflets 18a, 18b of the mitral valve 10. In some embodiments, only the second peaks 66 penetrate into the leaflets 18a, 18b. In other embodiments, some of the first peaks 64 also penetrate the leaflets 18a, 18b.

Referring to FIG. 8D, the inflatable member 32 may then be urged further along the proximal direction 80 such that the expandable member 34 is moved off of the inflatable member 32. The inflatable member 32 may be partially or completely deflated prior to urging the inflatable member 32 away from the expandable member 34. For example, the inflatable member 32 may remain partially inflated such that the partially inflated diameter is greater than a largest outer diameter of the safety catch 42 such that the expandable member 34 is able to slide over the catch 42.

Referring to FIG. 8E, following removal of the inflatable member 32, the expandable member 34 may contract due to biasing forces within the expandable member 34. As shown in FIG. 8F, contraction of the expandable member 34 tends to draw the leaflets 18a, 18b together, thereby reducing mitral valve regurgitation. In some embodiments, two or more expandable members 34 may be placed within a mitral valve according to the method illustrated in FIGS. 8A through 8F in order to further reduce regurgitation.

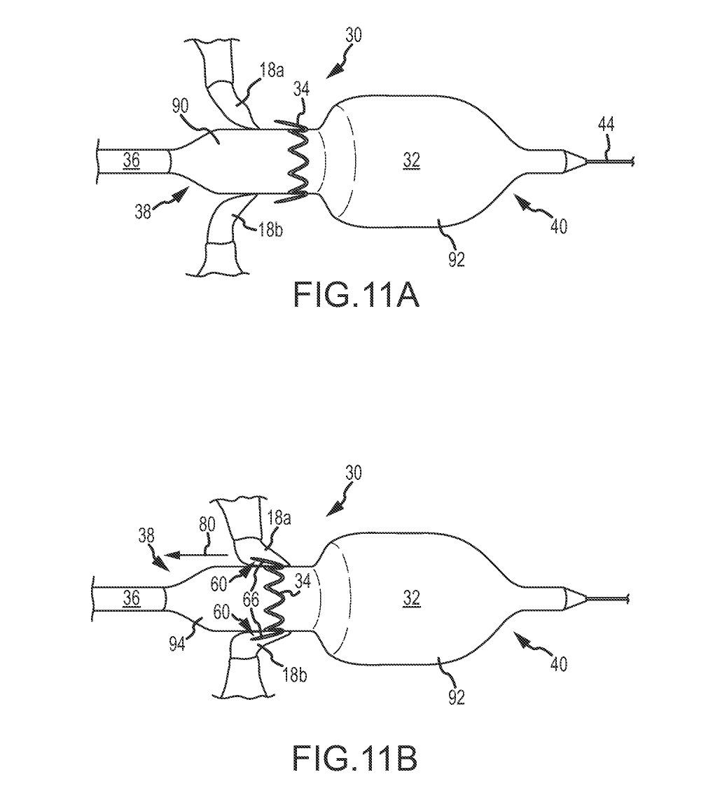

Referring to FIG. 9, in an alternative embodiment, the inflatable member 32 includes a first portion 90 and a second portion 92. The first portion 90 is located nearer the proximal end 38 and the second portion 92 is located nearer the distal end 40 of the inflatable member 32. The second portion 92 has an inflated diameter 94 that is larger than the inflated diameter 96 of the first portion. For example, the inflated diameter 94 of the second portion 92 may be between 1.3 and four times, or between about 1.4 and two times, the inflated diameter 96 of the first portion 90. Inasmuch as the left ventricle has a profile that is considerably larger than the mitral valve diameter a device may be used with a second portion 92 having diameter that is considerably larger than the ranges specified above.

The uninflated diameters of both the first portion 90 and second portion 92 may both be less than an inner diameter of the lumen of the catheter 36. In the illustrated embodiment, the second portion 92 also has a longer length 98 along the distal direction 48 than the length 100 of the first portion. For example, the inflated length 98 of the second portion 92 may be between 1.3 and four times, or between about 1.4 and two times the length 100 of the first portion. However, it will be appreciated that the left ventricle has a length that is considerably greater than the left atrium length and therefore a device may be used with a second portion 92 diameter that is considerably larger than the ranges specified above. The first portion 90 and second portion 92 may be in fluid communication with one another or may be isolated from one another and filled by means of separately controlled inflation channels extending through the catheter 36.

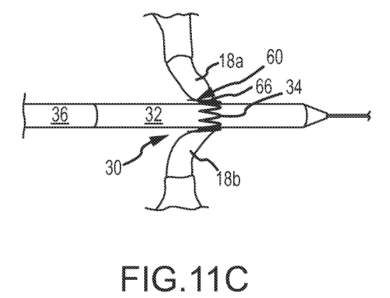

Referring to FIG. 10, in embodiments where the first portion 90 and second portion 92 are separate chambers, each portion 90, 92, may be coupled to a separate inflation line 54a, 54b coupled to the pressure source 52 and controlled by separate valves 56a, 56b, respectively to allow inflation medium to enter and leave the portions 90, 92. By selectively opening and closing one or both of the valves 56a, 56b, the diameters of the portions 90, 92 may be independently controlled in order to deploy the expandable member 34 according to the methods described hereinbelow.

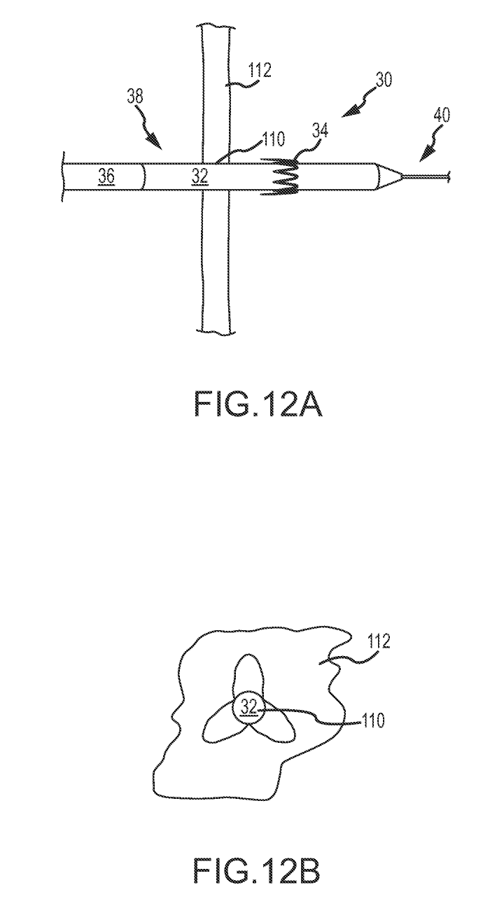

Referring to FIG. 11A, following insertion of the apparatus 30 into the mitral valve 10, such as is shown in FIG. 8A, the portions 90 and 92 may be inflated such that the diameter of the second portion 92 is substantially greater than that of the first portion 90. The difference in the diameters of the first portion 90 and second portion 92 may be sufficiently large to prevent the expandable member 34 from sliding toward the distal end 40.

Referring to FIG. 11B, the inflatable member 32 and expandable member 34 may then be drawn in the proximal direction 80 a sufficient distance that the projections 60 of the expandable member 34 penetrate the leaflets 18a, 18b of the mitral valve 10. The portions 90 and 92 may then be deflated as shown in FIG. 11C and the inflatable member 32 drawn in the proximal direction 80, leaving the expandable member in the position shown in FIG. 8F. In some instances, the second portion 92 can be used to push the projections 60 into the leaflets 18a and 18b prior to being deflated. This can ensure that the device 30 is securely engaged with the tissue to be brought together.

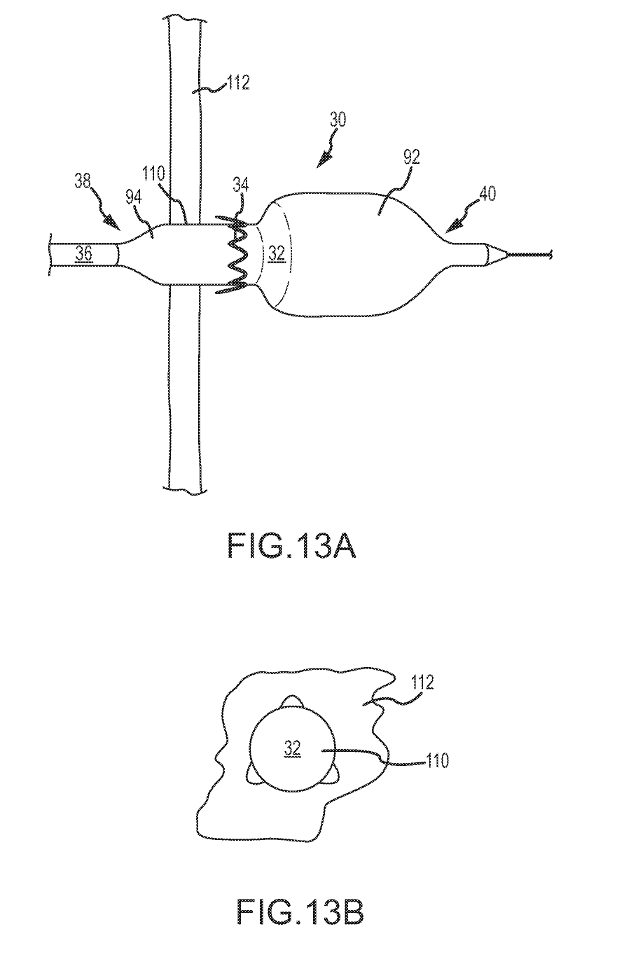

Referring to FIG. 12A through 15B, the apparatus 30 may also be used for associating other types of tissue. For example, the apparatus may be used to close a patent foramen ovale (PFO), a septal defect, or a puncture site in a wall of a body lumen, such as a blood vessel.

Referring to FIGS. 12A and 12B, an opening 110 in a wall 112 of tissue may be irregular in shape, particularly if formed as the result of accidental trauma. The wall 112 may represent the wall of a blood vessel, organ, the atrial or ventricular septum of the heart, or other structure. The inflatable member 32 and expandable member may be inserted through the opening 110, as shown in FIG. 12B, such that the distal end 40 and expandable member 34 are located on an opposite side of the wall 112 than the proximal end 38.

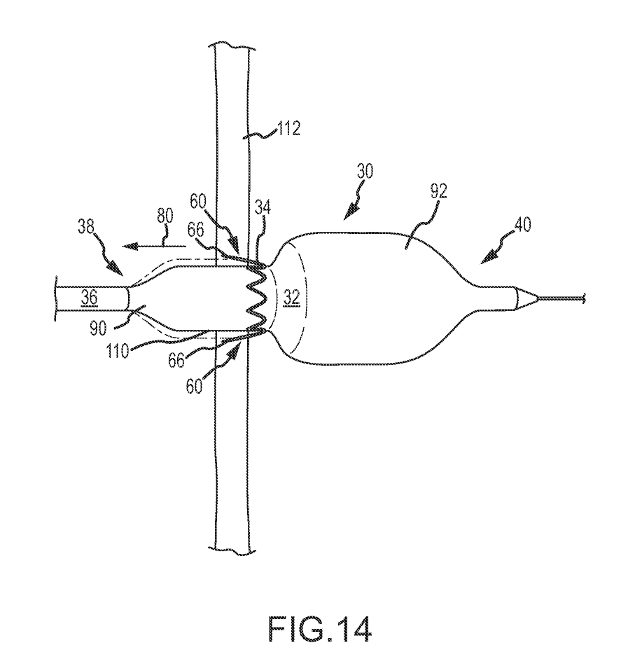

Referring to FIGS. 13A and 13B, the inflatable member 32 may then be inflated to substantially and/or partially occupy the entire opening 110 (e.g. between 70% and 100%, preferably greater than 90%, of the opening). Where the inflatable member 32 has a first portion 90 and second portion 92 with different inflated diameters, inflating the inflatable member may include inflating both portions 90 and 92.

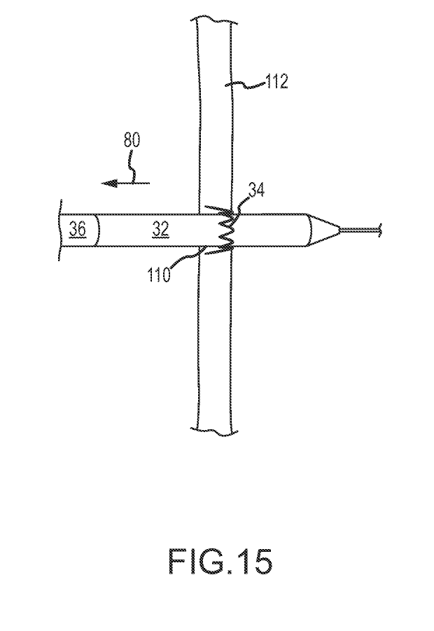

Referring to FIG. 14, the inflatable member 32 may then be urged in the proximal direction 80 such that the peaks 60 of the expandable member 34 are urged into the wall 112. The expandable member 34 may be the same as in other embodiments described herein. In some embodiments, where the expandable member is used for closing apertures other than the mitral valve, the projections 60, such as the second peaks 66, may be distributed along the entire circumference of the expandable member at regular intervals (e.g. between 15 and 45 degrees). In some embodiments, after the expandable member 34 is urged into the wall 112, the inflatable member 32 may be further inflated (as shown by the dotted lines in FIG. 14) in order to prevent bleeding through the opening 110.

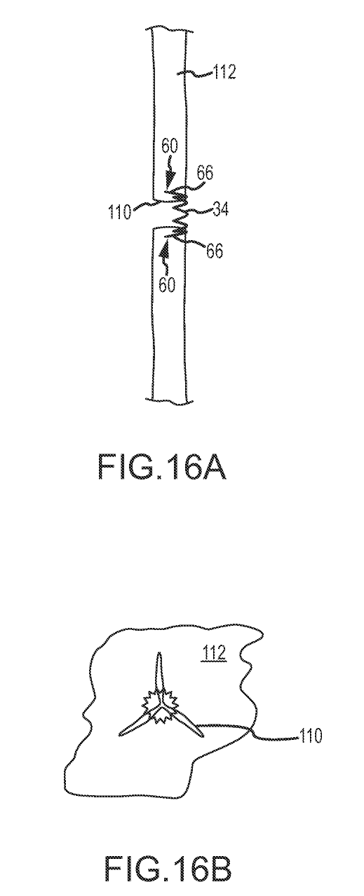

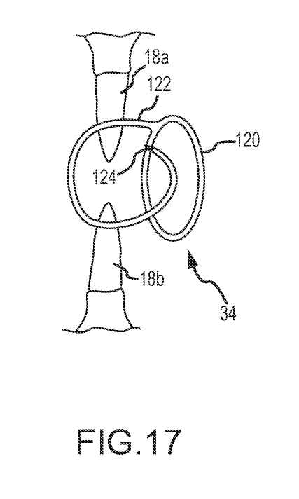

Referring to FIG. 15, after the expandable member 34 is implanted in the wall 112, the inflatable member 32 may then be deflated and withdrawn along the proximal direction 80 to leave the expandable member 34 in the configuration shown in FIGS. 16A and 16B. As is readily apparent, the expandable member 34 is able to significantly decrease the size of the opening 110, despite the irregular shape of the opening 110.

The expandable member 34, of the present invention can be made of a variety of materials, such as, but not limited to, those materials which are well known in the art of medical device manufacturing. Generally, the materials for the expandable member 34 can be selected according to the structural performance and biological characteristics that are desired. Materials well known in the art for preparing medical devices (e.g., endoprostheses), such as polymers and metals, can be employed in preparing the expandable member 34.

In one embodiment, the medical device can include a material made from any of a variety of known suitable materials, such as a shaped memory material (“SMM”) or superelastic material. For example, the SMM can be shaped in a manner that allows for restriction to induce a substantially tubular, linear orientation while within a delivery shaft (e.g., delivery catheter or encircling an expandable member), but can automatically retain the memory shape of the medical device once extended from the delivery shaft. SMMs have a shape memory effect in which they can be made to remember a particular shape. Once a shape has been remembered, the SMM may be bent out of shape or deformed and then returned to its original shape by unloading from strain or heating. SMMs can be shape memory alloys (“SMA”) or superelastic metals comprised of metal alloys, or shape memory plastics (“SMP”) comprised of polymers.

An SMA can have any non-characteristic initial shape that can then be configured into a memory shape by heating the SMA and conforming the SMA into the desired memory shape. After the SMA is cooled, the desired memory shape can be retained. This allows for the SMA to be bent, straightened, compacted, and placed into various contortions by the application of requisite forces; however, after the forces are released, the SMA can be capable of returning to the memory shape. The main types of SMAs are as follows: copper-zinc-aluminium; copper-aluminium-nickel; nickel-titanium (“NiTi”) alloys known as nitinol; and cobalt-chromium-nickel alloys or cobalt-chromium-nickel-molybdenum alloys known as elgiloy. The nitinol and elgiloy alloys can be more expensive, but have superior mechanical characteristics in comparison with the copper-based SMAs. The temperatures at which the SMA changes its crystallographic structure are characteristic of the alloy, and can be tuned by varying the elemental ratios.

For example, the primary material of the expandable member 34 can be of a NiTi alloy that forms superelastic nitinol. Nitinol materials can be trained to remember a certain shape, straightened in a shaft, catheter, or other tube, and then released from the catheter or tube to return to its trained shape. Also, additional materials can be added to the nitinol depending on the desired characteristic.

An SMP is a shape-shifting plastic that can be fashioned into the expandable member 34 in accordance with the present invention. When an SMP encounters a temperature above the lowest melting point of the individual polymers, the blend makes a transition to a rubbery state. The elastic modulus can change more than two orders of magnitude across the transition temperature (“Tₜᵣ”). As such, an SMP can be formed into a desired shape of expandable member 34 by heating it above the Tₜᵣ, fixing the SMP into the new shape, and cooling the material below Tₜᵣ. The SMP can then be arranged into a temporary shape by force and then resume the memory shape once the force has been applied. Examples of SMPs include, but are not limited to, biodegradable polymers, such as oligo(ε-caprolactone)diol, oligo(ρ-dioxanone)diol, and non-biodegradable polymers such as, polynorborene, polyisoprene, styrene butadiene, polyurethane-based materials, vinyl acetate-polyester-based compounds, and others yet to be determined. As such, any SMP can be used in accordance with the present invention.

Also, it can be beneficial to include at least one layer of an SMA and at least one layer of an SMP to form a multilayered body; however, any appropriate combination of materials can be used to form a multilayered medical device.

The expandable member 34 can be comprised of a variety of known suitable deformable materials, including stainless steel, silver, platinum, tantalum, palladium, cobalt-chromium alloys such as L605, MP35N, or MP20N, niobium, iridium, any equivalents thereof, alloys thereof, and combinations thereof. The alloy L605 is understood to be a trade name for an alloy available from UTI Corporation of Collegeville, Pa., including about 53% cobalt, 20% chromium and 10% nickel. The alloys MP35N and MP20N are understood to be trade names for alloys of cobalt, nickel, chromium and molybdenum available from Standard Press Steel Co., Jenkintown, Pa. More particularly, MP35N generally includes about 35% cobalt, 35% nickel, 20% chromium, and 10% molybdenum, and MP20N generally includes about 50% cobalt, 20% nickel, 20% chromium and 10% molybdenum.

Also, the expandable member 34 can include a suitable biocompatible polymer in addition to or in place of a suitable metal. The polymeric expandable member 34 can include a biocompatible material, such as biostable, biodegradable, or bioabsorbable materials, which can be either plastically deformable or capable of being set in the deployed configuration. If plastically deformable, the material can be selected to allow the medical device (e.g., stent) to be expanded in a similar manner using an expandable member so as to have sufficient radial strength and scaffolding and also to minimize recoil once expanded. If the polymer is to be set in the deployed configuration, the expandable member 34 can be provided with a heat source or infusion ports to provide the required catalyst to set or cure the polymer. Biocompatible polymers are well known in the art, and examples are recited with respect to the polymeric matrix. Thus, the expandable member 34 can be prepared from a biocompatible polymer.

Moreover, the expandable member 34 can include a radiopaque material to increase visibility during placement. Optionally, the radiopaque material can be a layer or coating any portion of the expandable member 34. The radiopaque materials can be platinum, tungsten, silver, stainless steel, gold, tantalum, bismuth, barium sulfate, or a similar material.

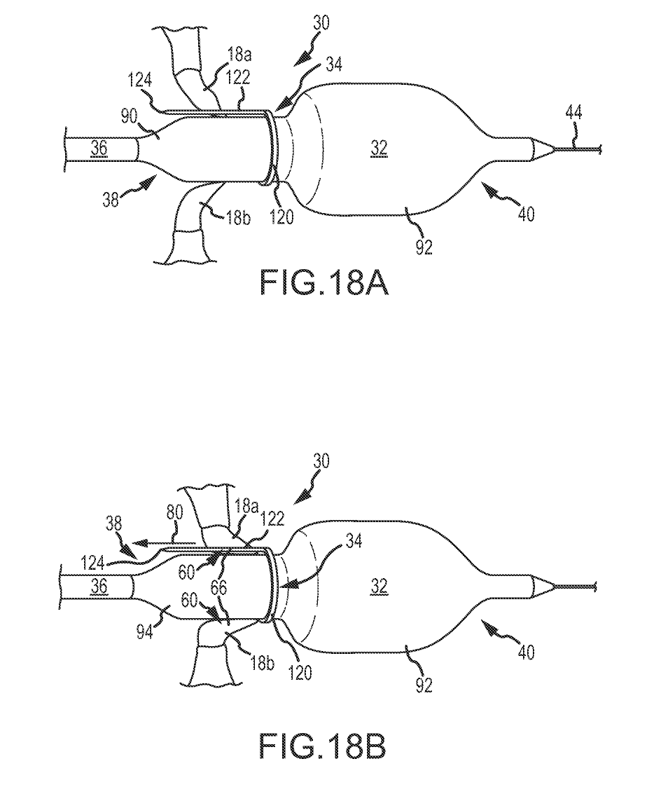

Referring to FIG. 17, in some embodiments the expandable member 34 may be embodied as a ring member 120 having a single projection 122 with a sharpened distal tip 124. The ring member 120 may include an elastic material that can expand upon inflation of the inflatable member 32. The ring member may also be formed of an undulating wire or other thin member in order to facilitate expansion upon inflation. The single projection 122 may have a relaxed of shape-memory shape that is curved in shape.

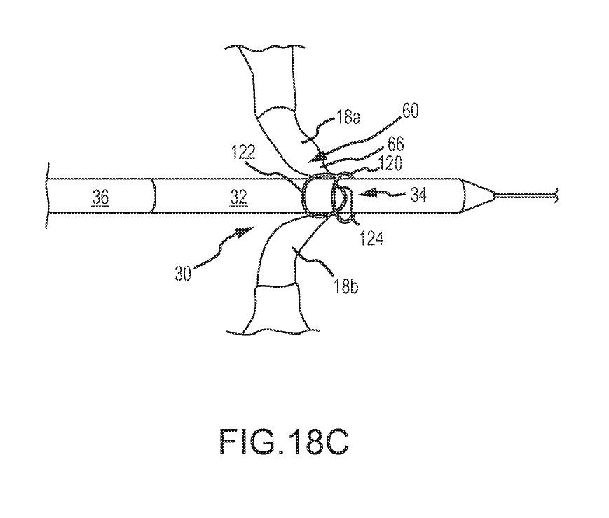

As shown in FIGS. 18A and 18B, the expandable member 34 of FIG. 17 may be placed on an inflatable member 32, such as any of the inflatable members illustrated herein. Upon inflation of the inflatable member 32, the projection 122 is urged into a straightened configuration such that the sharpened distal tip 124 may be urged in the distal direction 40 through one of the leaflets 18a. Referring to FIG. 18C, upon deflation of the inflatable member 32, the projection 122 is allowed to relax to its circular configuration, causing the sharpened distal tip 124 to be driven through the opposing leaflet 18b. The inflatable member 32 may then be removed.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus for reducing a distance between adjacent tissue interfaces comprising:
an inflatable member;
an elastically expandable member encircling the inflatable member, the elastically expandable member comprising a thin circuitous member defining a base line lying in a plane and circumscribing the inflatable member, the thin circuitous member bent to define a first plurality of peaks extending a first distance measured from the base line parallel to the inflatable member and a second plurality of peaks extending a second distance measured from the base line parallel to the inflatable member, the second distance being substantially greater than the first distance.

2. The apparatus of claim 1, wherein the second distance is between 1.2 and 1.6 times the first distance.

3. The apparatus of claim 1, wherein the second plurality of peaks extend away from the inflatable member with distance from the base line.

4. The apparatus of claim 3, wherein the second plurality of peaks extend away from the first plurality of peaks with distance from the base line.

5. The apparatus of claim 1, wherein a distal end of the second plurality of peaks is sharpened.

6. The apparatus of claim 1, wherein a distal end of the second plurality of peaks includes at least one beveled surface.

7. The apparatus of claim 1, wherein the thin circuitous member comprises nitinol.

8. The apparatus of claim 1, further comprising a lumen extending through the inflatable member and a guide wire positioned within the lumen.

9. The apparatus of claim 1, further comprising a catch secured adjacent the inflatable member and having a diameter greater than that of an adjacent portion of the elastically expandable member when the inflatable member is not inflated.

10. The apparatus of claim 1, wherein the inflatable member includes a first portion and a second portion, the elastically expandable member encircling the first portion, wherein the second portion has an inflated diameter substantially greater than the first portion.

11. The apparatus of claim 9, wherein the inflatable member has a distal end and a proximal end, the proximal end being configured to receive an inflation medium; and wherein the first portion is positioned between the second portion and the proximal end.

12. An apparatus for reducing a distance between adjacent tissue interfaces comprising:
an inflatable member having a first portion and a second portion, the second portion having an inflated diameter substantially greater than the first portion; and
an elastically expandable member encircling the first portion of the inflatable member, the elastically expandable member defining at least one peak.

13. The apparatus of claim 12, wherein the first portion is adapted to receive an inflation medium.

14. The apparatus of claim 13, wherein the first portion is adapted to secure to a catheter.

15. A method for associating first and second tissue edges comprising:
inserting an inflatable member between the first and second tissue edges, the inflatable member having a distal end and a proximal end, the first and second tissue edges being located between the distal end and the proximal end, the inflatable member further having an elastically expandable member encircling the inflatable member between the distal end and the first and second tissue edges, the elastically expandable member defining a plurality of peaks;
inflating the inflatable member;
urging the distal end toward the first and second tissue edges such that at least a portion of the plurality of peaks engage tissue adjacent at least one of the first and second tissue edges; and
withdrawing the inflatable member from between the first and second tissue edges.

16. The method of claim 15, further comprising deflating the inflatable member prior to withdrawing the inflatable member from between the first and second tissue edges.

17. The method of claim 15, wherein the inflatable member includes a first portion and a second portion, the second portion located closer to the proximal end than the first portion and the elastically expandable member encircling the first portion; and wherein inflating the inflatable elastically expandable member comprises inflating the first and second portions such that the second portion has an inflated diameter substantially greater than the first portion.

18. The method of claim 15, wherein the elastically expandable member comprises a thin circuitous member bent to define the plurality of peaks.

19. The method of claim 18, wherein the thin circuitous member comprises nitinol.

20. The method of claim 15, wherein the first and second tissue edges form part of at least one of a patent foramen ovale, a septal defect, and a puncture site in a wall of a body lumen.

21. The method of claim 15, wherein the first tissue edge is formed on a first leaflet of a mitral valve and the second tissue edge is formed on a second leaflet of the mitral valve.

22. An assembly for associating first and second tissue edges comprising:
a catheter defining a catheter lumen;
an inflatable member positionable within the lumen and defining a guide wire lumen, the inflatable member having a first portion and a second portion, the second portion having an inflated diameter substantially greater than the first portion;
an elastically expandable member encircling the first portion of the inflatable member, the elastically expandable member defining a plurality of peaks; and
a guide wire extending through catheter lumen and the guide wire lumen.

23. The assembly of claim 22, wherein the plurality of peaks comprises a first plurality of peaks and a second plurality of peaks and wherein the expandable member comprises a thin circuitous member defining a base line lying entirely in a plane and circumscribing the inflatable member, the thin circuitous member being bent to define the first plurality of peaks and the second plurality of peaks, the first plurality of peaks extending a first distance measured from the base line parallel to the inflatable member and the second plurality of peaks extending a second distance measured from the base line parallel to the inflatable member, the second distance being substantially greater than the first distance.

24. The assembly of claim 23, wherein the second distance is between 1.2 and 1.6 times the first distance.

25. The assembly of claim 23, wherein the second plurality of peaks extend away from the inflatable member with distance from the base line.

26. The assembly of claim 23, wherein a distal end of the second plurality of peaks includes at least one beveled surface.

27. An apparatus for reducing a distance between adjacent tissue interfaces comprising:
an elastically expandable member, the elastically expandable member comprising a thin circuitous loop defining a base line lying in a plane, the thin circuitous member being bent to define a first plurality of peaks extending a first distance measured from the base line and a second plurality of peaks extending a second distance measured from the base line, the second distance being substantially greater than the first distance.

28. The apparatus of claim 27, wherein the second distance is between 1.2 and 1.6 times the first distance.

29. The apparatus of claim 28, wherein the second plurality of peaks extend away from the first plurality of peaks with distance from the base line.

30. The apparatus of claim 27, wherein a distal end of the second plurality of peaks is sharpened.

31. The apparatus of claim 27, wherein a distal end of the second plurality of peaks includes at least one beveled surface.

32. The apparatus of claim 27, wherein the thin circuitous member comprises nitinol.
